(19) United States
(12) Patent Application Publication
(10) Pub. No.: US 2026/0174713 A1
(43) Pub. Date: Jun. 25, 2026

(54) METHODS FOR TREATING BIOFILMS

(71) Applicant: NEXT SCIENCE IP HOLDINGS PTY LTD, Chatswood, New South Wales (AU)

(72) Inventor: Matthew MYNTTI, St. Augustine, FL (US)

(73) Assignee: NEXT SCIENCE IP HOLDINGS PTY LTD, Chatswood, New South Wales (AU)

(21) Appl. No.: 19/132,437

(22) PCT Filed: Nov. 28, 2023
(86) PCT No.: PCT/US2023/081247

§ 371 Date: May 23, 2025

(51) Int. Cl. A61K 31/185; A61K 47/12

(52) U.S. Cl. CPC A61K 31/185; A61K 47/12

(57) ABSTRACT

Existing biofilms on a range of surfaces, including previously implanted medical devices and human skin, can be treated via application of an acidic liquid which has an effective solute concentration of from 0.3 to 1.4 Osm/L and a pH of from 3.7 to 4.2.

### CROSS REFERENCE TO RELATED APPLICATION

This application claims priority to U.S. patent appl. No. 63/428,165 filed on Nov. 28, 2022, which is incorporated herein by reference in its entirety.

### BACKGROUND INFORMATION

Microbes are found virtually everywhere, often in high concentrations, and are responsible for a significant amount of disease and infection.

Bacteria present special challenges because they can exist in a number of forms (e.g., planktonic, spore and biofilm) and their self-preservation mechanisms complicate or even confound efforts to treat and/or eradicate them. For example, bacteria in biofilms or spores typically are down-regulated (sessile) and not actively dividing, which makes them resistant to attack by those antibiotics and antimicrobials which attack bacteria during cell division.

In a biofilm, which can form on any surface that is or that can become moist, bacteria interact with and adhere to surfaces and form colonies which facilitate continued growth. The bacteria produce exopolysaccharide (EPS) and/or extracellular polysaccharide (ECPS) macromolecules that keep them attached to the surface and form a protective barrier. Protection most likely can be attributed to the small diameters of the flow channels in the matrix, which restrict the size of molecules that can reach the underlying bacteria, and consumption of biocides through interactions with portions of the EPS/ECPS macromolecular matrix and bacterial secretions and waste products contained therein.

Artificial joints and other orthopedic hardware are particularly susceptible to formation of biofilms due to the lack of blood flow to the site of infection. Despite pre-, peri- and post-surgical efforts to protect against bacterial growth in and around such orthopedic hardware, periprosthetic joint infection (PJI) remains a common occurrence and the source of significant costs to both the patient and his or her healthcare provider(s).

If efforts like intravenous introduction of antibiotics do not work against a PJI, the patient is subjected to a lengthy and costly in-patient regimen typically involving removal of the impacted hardware, debridement of surrounding tissue and bone, and, after flooding the infected area with antibiotics, a second implantation surgery, i.e., a so-called revision. Revisions are known to involve significant costs and non-trivial patient morbidity and mortality.

International patent appl. publ. WO 2022/081737 teaches methods of preventing formation of biofilms in or around surgical cavities via introduction thereto prior to post-surgical approximation of the surgical site wound. The publication does not describe its compositions as being used to treat an existing, i.e., already formed, biofilm.

Ridding surfaces of microbes and especially bacteria, particularly bacteria in the form of biofilms, is desirable for many reasons, including in the treatment of infection during revision arthroplasty. In addition, biofilms are known to be a critical element in certain skin diseases, including atopic dermatitis (eczema/atopic eczema) and acne vulgaris; see, e.g., M. Brandwein et al., “Microbial biofilms and the human skin microbiome,” NPJ Biofilms Microbiomes 2:3 (2016).

Compositions that are compatible with human tissue and that are able to treat, reduce, eliminate, etc., bacteria, particularly bacteria in biofilm form on inanimate surfaces including on previously implanted medical devices or on the skin, are highly desirable. Such compositions that do not need to be rinsed from the skin or body after contact, or from an inanimate surface prior to, during, or after contact with the skin or body, or from a surgical site, are particularly desirable.

### SUMMARY

Provided herein are methods for treating existing biofilms, wherever they may be located, using aqueous compositions, preferably sterile aqueous compositions.

Typical compositions employed have calculated effective solute concentrations of from 0.3 to 1.4 Osm/L, particularly from 0.3 to 0.7 Osm/L. Such compositions preferably are acidic, e.g., 3.7≤pH≤4.2.

The compositions can be directly applied to human tissue and to existing biofilms thereon, to the body, both to and in a surgical cavities, and to a medical device implanted in the body. Advantageously, the composition need not be diluted or removed, in part or in whole, after application or introduction.

In certain embodiments, the compositions are free of materials, other than those making up composition, having active antimicrobial properties including, but not limited to, antibiotics. Alternatively, a material or compound having active antimicrobial properties can be included in embodiments of the composition.

A PJI can be treated by applying a treatment composition to previously implanted medical hardware having thereon a biofilm, optionally also to surrounding tissue. The application in this method occurs not only after implantation of the hardware but also after approximation of the surgical wound cavity made during/for that implantation. In preferred embodiments, the treating composition is not diluted nor wholly removed (via suction) from the wound cavity made to expose the previously implanted medical device prior to approximation thereof.

Also described are methods of treating conditions in/on human tissue, particularly skin, such as atopic dermatitis (eczema) and acne vulgaris, in which a treating composition is administered to a dermal area where the condition is located (“affected area”) by any technique whereby the composition is applied or introduced, non-limiting examples of which include spraying, use of applicators, bulb syringes, cotton balls, pads, etc. Also contemplated are methods for prophylactically treating these types of conditions by administering a composition to a previously affected area of a patient in need thereof and/or to areas of the body known to be susceptible to such conditions.

The compositions can be applied to tissue including a surgical wound cavity or the area immediately surrounding such cavity (e.g., skin), i.e., that area around a wound cavity that might come into contact with a composition used in accordance with the methods of the aforementioned WO 2022/081737. Particularly in the cases of introduction into surgical wound cavities, the composition in the present methods encounters an established biofilm as opposed to merely inhibiting formation of biofilm by killing planktonic bacteria or nascent biofilms.

Other aspects of the invention will be apparent to the ordinarily skilled artisan from the detailed description that follows. To assist in understanding that description, certain definitions are provided immediately below, and these are intended to apply throughout unless the surrounding text explicitly indicates a contrary intention:
“comprising” means including, but not limited to, the listed ingredients or steps;
“consisting of” means including only the listed ingredients (or steps) and minor amounts of inactive additives or adjuvants;
“room temperature” means 20° to 25° C.;
“body temperature” means the average temperature of a mammal ±1.5° C., for example, ˜35° to ˜38° C. for a North American human, ˜37° to ˜40° C. for a canine, etc.;
“treat” (or variants such as “treating”) means
   with respect to a biofilm located on an inanimate surface such as an implanted medical device, to provide a reduction in bioburden thereon, or
   with respect to a condition associated with or resulting from a biofilm on human tissue, any one or more of to reduce, slow, attenuate, inhibit, stop, eliminate, and reverse any one or more of the symptoms, effects, characteristics, features, and clinical manifestations associated with the condition;
“patient in need thereof” is a mammal with one or more conditions associated with biofilms on tissue, particularly skin, such as atopic dermatitis (eczema/atopic dermatitis) and acne vulgaris, and in the case of prophylactic treatment, who had and/or is susceptible to one or more such conditions,
“in situ” means situated in the existing place or position,
“in situ treatment of an implanted medical device” means treating a previously-implanted medical device in the place or position in which it was implanted;
“skin” as used herein excludes a surgical wound cavity and the area of skin immediately surrounding such cavity, i.e., that area around a wound cavity that might come into contact with a composition used in accordance with the methods of WO 2022/081737;
“polyacid” means a compound having at least two carboxyl groups and specifically includes dicarboxylic acids, tricarboxylic acids, etc.;
“pH” means the negative value of the base 10 logarithm of [H⁺] as determined by an acceptably reliable measurement method such as a properly calibrated pH meter, titration curve against a known standard, or the like;
“pCₐ” means the negative value of the base 10 logarithm of a particular compound's acid dissociation constant;
“buffer” means a compound or mixture of compounds having an ability to maintain the pH of a solution to which it is added within relatively narrow limits;
“buffer precursor” means a compound that, when added to a mixture containing an acid, results in a buffer;
“electrolyte” means a compound that exhibits some dissociation when added to water;
“purified water” means water having a bacterial count and a level of endotoxins below those in tap water, well water, or spring water, either as-is or after a treatment such as softening or ion exchange;
“pharmaceutical grade” means a compound which meets a chemical purity standard established by a national or regional pharmacopeia;
“medication” means a substance which provides a therapeutic benefit to a treated subject;
“viscosifier” means a compound that decreases the speed at which a liquid spreads while still permitting that liquid some degree of flow at room temperature or higher;
“effective solute concentration” is a measurement of the colligative property resulting from the number of moles of molecules (from nonelectrolyte) or ions (from electrolytes) present in a given volume solution, often presented in units of osmoles per liter;
“calculated effective solute concentration” means the effective solute concentration, at room temperature and a given pH, of a composition determined by assuming full dissolution of solute(s) as opposed to through/by measurement of a colligative property;
“sterile,” when used in connection with a liquid composition and/or a container for such a liquid, means one which has been treated so as to kill any living organisms contained therein;
“substituted” means containing a heteroatom or functionality (e.g., hydrocarbyl group) that does not interfere with the intended purpose of the group in question;
“approximate,” when used in connection with a surgical procedure, means the process whereby a surgical wound is closed;
“wound cavity” means the area of a body which, although typically covered by the dermis, is capable of being contacted by a liquid introduced from an external source;
“microbe” means any type of microorganism including, but not limited to, bacteria, viruses, fungi, viroids, and prions;
“bioburden” means microbes and/or a substance produced, excreted or resulting from the presence of microbes;
“antimicrobial agent” means a substance having the ability to cause greater than a 90% (1 log) reduction in the number of one or more microbes;
“active antimicrobial agent” means an antimicrobial agent that is effective only or primarily during the active parts of the lifecycle, e.g., cell division, of a microbe;
“dwell time” means the amount of time that a composition is allowed to contact a surface and/or a microbe on such a surface; and
“healthcare” means involved in or connected with the maintenance or restoration of the health of the body or mind.

Throughout this document, unless the surrounding text explicitly indicates a contrary intention, all values given in the form of percentages are w v, i.e., grams of solute per liter of composition and pH values are those which can be obtained from any of a variety of potentiometric techniques employing a properly calibrated electrode. The terms “invention,” “present invention,” and the like refer only to the particular embodiment(s) immediately mentioned and are not to be construed as limiting the overall contributions to the art described herein, nor generally or specifically limiting with regard to the several individual advances in the art described.

The relevant portion(s) of any specifically referenced patent and/or published patent application are incorporated herein by reference.

### DETAILED DESCRIPTION OF ILLUSTRATIVE EMBODIMENTS

The compositions utilized in the inventive methods are described first in terms of its properties and components and then in terms of particular uses. Multiple compositions are described and, unless the context indicates otherwise, the terms “composition” and “compositions” refer to all embodiments described herein.

The composition includes solvent and solute components.

The solvent component is primarily water, typically purified water. Relative to the total volume of the solvent component, purified water constitutes at least 95%, often at least 97%, and typically at least 99% (all w/v) thereof. On a per liter basis, a composition includes from ˜925 to ˜975, commonly from ˜937 to ˜972, more commonly from ˜950 to ˜970, and typically 960±5 mL purified water. A preferred solvent component is 100% purified water.

Although not preferred, the solvent component can include small volumes of one or more organic liquids. A compilation of potentially useful organic liquids is provided in, for example, U.S. Pat. No. 10,021,876, with those being listed on the U.S. Food and Drug Administration inactive ingredients list (https://www.fda.gov/media/72482 download, link active as of filing date of this application), non-limiting examples of which include ethanol and propylene glycol, being preferred. Where more than one organic liquid is included, each should be unreactive toward the other(s). The organic liquids can constitute no more than 5%, preferably no more than 3%, and most preferably no more than 1% (all w/v) of the solvent component.

Each sub-component of the solute component preferably is provided in pharmaceutical grade form, particularly where the composition is to be used in the treatment of a mammalian, typically human, patient.

The composition is acidic, which means that at least one of the sub-components of the solute component is an acid. Preferred acids are those which have relatively high pKₐ values, i.e., are not considered to be strong acids.

Examples of potentially useful weak acids include monoprotic acids such as formic acid, acetic acid and substituted variants (e.g., hydroxyacetic acid, chloroacetic acid, dichloroacetic acid, phenylacetic acid, and the like), propanoic acid and substituted variants (e.g., lactic acid, pyruvic acid, and the like), any of a variety of benzoic acids (e.g., mandelic acid, chloromandelic acid, salicylic acid, and the like), glucuronic acid, and the like; diprotic acids such as oxalic acid and substituted variants (e.g., oxamic acid), butanedioic acid and substituted variants (e.g., malic acid, aspartic acid, tartaric acid, citramalic acid, and the like), pentanedioic acid and substituted variants (e.g., glutamic acid, 2-ketoglutaric acid, and the like), hexanedioic acid and substituted variants (e.g., mucic acid), butenedioic acid, iminodiacetic acid, phthalic acid, and the like; triprotic acids such as citric acid, 2-methylpropane-1,2,3-tricarboxylic acid, benzenetricarboxylic acid, nitrilotriacetic acid, and the like; tetraprotic acids such as prehnitic acid, pyromellitic acid, and the like; and even higher degree acids (e.g., penta-, hexa-, heptaprotic, etc.). Where a tri-, tetra-, or higher acid is used, one or more of the carboxyl protons can be replaced by cationic atoms or groups (e.g., alkali metal ions), which can be the same or different.

Citric acid constitutes a preferred acid because mammalian bodies have such familiarity with and tolerance toward it due to its use and regeneration as part of the Krebs cycle. Those solute components which include citric acid, particularly those which have citric acid as their sole acid, are preferred.

The amount of any given acid employed can be determined from the target pH of a given composition and the pKₐ value(s) of the chosen acids in view of the type and amounts of compound(s), if any, utilized to achieve the desired effective solute concentration.

Both to ensure that the pH of the composition is not too low and also to increase its effective solute concentration, the solute component also includes a conjugate base of at least one of the foregoing weak acids. Although not required, use of conjugate base(s) of the particular acid(s) employed is preferable.

Upon dissociation, conjugate bases, e.g., salt(s), of one or more of the acid(s) increase the effective amount of solutes in the composition without greatly impacting the molar concentration of hydronium ions while, simultaneously, act to buffer the pH of the composition. The identity of the countercation portion of the salt(s) is not believed to be particularly critical, with common examples including ammonium ions and alkali metals, with the latter being preferred countercations.

Where a conjugate base of polyacid is used, all or fewer than all of the H atoms of the carboxyl groups can be replaced with cationic atoms or groups, which can be the same or different. For example, mono-, di- and trisodium citrate all constitute potentially useful buffer precursors, whether used in conjunction with citric acid or another organic acid. However, because trisodium citrate has three available basic sites, it has a theoretical buffering capacity up to 50% greater than that of disodium citrate (which has two such sites) and up to 200% greater than that of sodium citrate (which has only one such site).

Like the acid(s) described above, the amount of conjugate base(s) can be determined based on the desired composition pH and effective solute concentration.

Many organic acids and their conjugate bases can be provided in either anhydrous or hydrate forms. The particular form of these materials does not impact utility or efficacy. Any water of hydration in the solute(s) merely becomes part of the solvent component.

The amounts of acid(s) and conjugate base(s) included in the solute component are added at levels that provide two important compositional characteristics, neither of which depends on the particular materials that provide them.

The first such characteristic is pH. The present composition has a pH of from 3.7 to 4.2. A composition which has an even lower pH is quite likely to be even more effective in terms of disrupting EPS/ECPS macromolecules and in killing bacteria; however, this increased efficacy comes at a cost of decreased biocompatibility. Conversely, a composition having a pH>4.2 would have even greater biocompatibility, albeit at the cost of lower efficacy.

Within the permitted pH range, a pH of from 3.85 to 4.05 is preferred, with pH=3.95±0.1 or even ±0.05 being particularly preferred. Preferred pH values include 3.7, 3.8, 3.9, 4, 4.1 and 4.2.

The second important compositional characteristic is effective solute concentration, which induces a sufficient osmotic pressure differential across a bacterium's cortical membrane to lead to lysis. This ability to induce an osmotic pressure differential is independent of the particular identity or nature of individual compounds (or their dissociation products) of the solute component, although smaller molecules and ions are generally more effective than larger ones due to ease of transport across cortical membranes and solvent capacity (i.e., the ability to (typically) include more small molecules in a given amount of solvent component than an equimolar amount of larger molecules).

The present composition has a calculated effective solute concentration of from ˜300 to ˜1400 mOsm/L, including a preferred range of from ˜300 to ˜1000 mOsm/L including from ˜300 to ˜700 mOsm/L. (A composition which has an effective solute concentration greater than ˜1400 mOsm/L could be even more effective in terms of lethality toward bacteria, albeit at a cost of decreased biocompatibility, specifically, tissue inflammation.) Within the foregoing general ranges, preferred ranges include ˜325 to ˜1325, ˜350 to ˜1275, ˜375 to ˜1200, ˜400 to ˜1150, ˜450 to ˜1100, ˜325 to ˜975, ˜350 to ˜900, ˜375 to ˜800, ˜400 to ˜750, ˜450 to ˜700, ˜325 to ˜675, ˜350 to ˜650, ˜375 to ˜600, ˜400 to ˜590, ˜450 to ˜700, ˜300 to ˜590, ˜350 to ˜450, ˜350 to ˜500, ˜350 to ˜590, ˜400 to ˜580, ˜450 to ˜575, ˜450 to ˜680, ˜460 to ˜650, and ˜470 to ˜635 mOsm/L. A preferred overall range is from 450 to 675 mOsm/L, and preferred calculated effective solute concentration target values include 300, 325, 350, 375, 400, 425, 450, 475, 500, 525, 550, 575, 600, 625, 650, 675, and 700 mOsm/L.

(Calculated effective solute concentration values constitute theoretical maxima based on full dissociation of all solutes. However, at the foregoing concentration ranges, these maxima are theoretical due to an increasing potential for reassociation of previously dissociated solutes. Nevertheless, because of the ease of the calculations involved and the availability of free online calculation tools, calculated effective solute concentration is a preferred way to discuss and consider the concept. For example, 0.1 M NaCl has a calculated effective solute concentration value of 200 mOsm/L (i.e., 0.1 mole each of Na⁺ and Cl⁻ ions at full dissociation.)

Effective solute concentration, itself a colligative property, can be determined by any of a variety of colligative property measurement techniques such as vapor pressure lowering, boiling point elevation, freezing point depression, and membrane osmometry. (Because of their impact on properties such as boiling point and freezing point, where a particular composition happens to include one or more organic liquids, a tested composition which includes an equivalent volume of purified water in place of the organic liquid(s) is used when performing one of the foregoing techniques so as to determine effective solute concentration.)

Preferred ranges of effective solute concentration as measured by freezing point depression include ˜250 to ˜1300, ˜275 to ˜1250, ˜300 to ˜1200, ˜325 to ˜800, ˜350 to ˜725, ˜250 to ˜600, ˜275 to ˜575, ˜300 to ˜550, ˜325 to ˜500, ˜350 to ˜425, ˜450 to ˜600, ˜300 to ˜500, ˜250 to ˜450, ˜350 to ˜500, and ˜450 to ˜535 mOsm/L. A preferred measured range is from 350 to 550 mOsm/L.

Using citric acid and a citrate that includes three alkali metal ions as an exemplary acid and conjugate base pair, acceptable values for the aforedescribed compositional characteristics can be achieved (or at least approached, to permit achievement via the type of minor modification described below) using from 25 to 40 g/L citric acid and from 30 to 45 g/L of a citrate. Where anhydrous citric acid and trisodium citrate dihydrate are utilized, a preferred embodiment (for peritoneal usage) can be provided from 30 to 35 g/L of the acid and 34 to 38 g/L of the citrate, while another preferred embodiment (for joint usage) can be provided from 33 to 38 g/L of the acid and 37 to 42 g/L of the citrate.

Importantly, the particular species of acid and citrate discussed in the preceding paragraph need not be utilized. An ordinarily skilled artisan desiring to use a hydrate version of the acid, an anhydrous version of the citrate, a citrate having fewer than three alkali metal atoms (i.e., mono- or disodium citrate) readily can calculate amounts of each that will provide compositions having acceptable values for the aforedescribed compositional characteristics.

The upper limit of the effective solute concentration can be impacted by the area of the body in which it is intended for use. For example, some studies have indicated that compositions having effective solute concentrations of ˜600 mOsm/L are better tolerated than lower concentration solutions when used in and around a joint, e.g., a shoulder, yet other studies have indicated that compositions having effective solute concentrations above ˜600 mOsm/L can cause irritation and swelling in the human peritoneal cavity.

In preferred embodiments the solute component also includes one or more surface active agents that bear some type of ionic charge. Of these, anionic and cationic surfactants are preferred over zwitterionic surfactants. A composition preferably does not include surfactant types that are incompatible, i.e., anionic with cationic or zwitterionic with either anionic or cationic. Cationic surfactants are preferred in those methods not involving portions of the composition remaining in the body, e.g., treatment of removed devices, hardware, etc. For in situ treatment mryhofd, anionic surfactants are preferred, and cationic and/or zwitterionic surfactants are preferably excluded.

Smaller molecules generally are preferred over larger sized surfactants. The size of side-groups attached to the polar head can influence the efficacy of ionic surfactants, with larger sized groups and more side groups on the polar head potentially decreasing its efficacy.

Potentially useful anionic surfactants include, but are not limited to, ammonium lauryl sulfate, dioctyl sodium sulfosuccinate, perfluorobutanesulfonic acid, perfluorononanoic acid, perfluorooctanesulfonic acid, perfluorooctanoic acid, potassium laurylsulfate, sodium dodecylbenzenesulfonate, sodium laureth sulfate, sodium lauroyl sarcosinate, sodium myreth sulfate, sodium pareth sulfate, sodium stearate, sodium chenodeoxycholate, N-lauroylsarcosine sodium salt, lithium dodecyl sulfate, 1-octanesulfonic acid sodium salt, sodium cholate hydrate, sodium deoxycholate, sodium dodecyl sulfate (SDS, also called sodium lauryl sulfate (SLS)), sodium glycodeoxycholate, and the alkyl phosphates set forth in U.S. Pat. No. 6,610,314. (Although sodium is used as the countercation in most of the foregoing exemplary anionic surfactants, other alkali metal ions can be used in its place.) SDS is a particularly preferred option.

Potentially useful cationic surfactants include, but are not limited to, cetylpyridinium chloride (CPC), cetyl trimethylammonium chloride, benzethonium chloride, 5-bromo-5-nitro-1,3-dioxane, dimethyldioctadecylammonium chloride, cetrimonium bromide, dioctadecyldimethylammonium bromide, tetradecyltrimethyl ammonium bromide, benzalkonium chloride (BZK), hexadecylpyridinium chloride monohydrate and hexadecyltrimethylammonium bromide.

Potentially useful zwitterionic surfactants include sulfonates (e.g. 3-[(3-cholamidopropyl)dimethylammonio]-1-propanesulfonate), sultaines (e.g. cocamidopropyl hydroxysultaine), betaines (e.g. cocamidopropyl betaine), and phosphates (e.g. lecithin).

Although not preferred as sole surface active agents, nonionic surfactant(s) can be included with one of the other types of surfactants.

For other potentially useful surface active materials, the interested reader is directed to any of a variety of other sources including, for example, U.S. Pat. Nos. 4,107,328, 6,953,772, 7,959,943, and 8,940,792.

The amount(s) of surfactant(s) included is limited to some extent by the target effective solute concentration and compatibility with other subcomponents of the solute component. The total amount of surfactant present in the composition can range from ˜0.07 to ˜0.19% (w/v), typically ˜0.075 to ˜0.15% (w v), preferably 1±0.25 g/L or 0.95±0.2 g/L.

If the acid(s), conjugate base(s) and surfactant(s) do not provide a desired effective solute concentration, one or more electrolytes, particularly ionic compounds (salts), can be added; see, e.g., U.S. Pat. No. 7,090,882, for a list of potentially useful electrolytes.

Not preferred but permissible in the solute component is one or more inactive ingredients (additives) approved by the U.S. Food & Drug Administration; see above.

A typical manner of making a composition involves adding the solute sub-components, either separately or as an admixture, to the solvent component (or to the water sub-component of the solvent component, followed by addition of the organic liquid(s)). This addition can be done with the benefit of one or both of stirring and heating of the mixing container.

If assurance of a targeted pH range is considered important, once the solute component has been added to the solvent component, very small aliquots of a concentrated acid (e.g., 1M HCl) or concentrated base (e.g., 1M KOH) can be used to lower or raise the composition's pH into the targeted range.

The following table provides an ingredient list for providing exemplary compositions according to the present invention, with amounts being given in grams.

TABLE 1
Formulations for exemplary compositions

| | | Amount, | Amount, |
|---|---|---|---|
| | Preferred species | generally | preferred |
| organic acid | citric acid | 30-35 | 31-34 |
| conjugate base of | citrate (e.g., | 30-40 | 34-38 |
| organic acid | trisodium citrate) | | |
| ionic surfactant | SDS | 0.85-1.15 | 0.9-2.1 |
| Water | USP water | 950-975 | 955-970 |

Various embodiments of the present invention have been provided by way of example and not limitation. As evident from the foregoing tables, general preferences regarding features, ranges, numerical limitations and embodiments are, to the extent feasible and as long as not interfering or incompatible, envisioned as being capable of being combined with other such generally preferred features, ranges, numerical limitations and embodiments.

The composition can be packaged in sterile form, i.e., its container having been subjected to sufficient heat, radiation, etc., so as to render the composition sterile (aseptic). Typical containers include bags and bottles of a type similar to those used to deliver liquids such as saline solutions in surgical theaters.

In cases where human tissue is being treated, and especially where a condition associated with biofilms on human skin such as atopic dermatitis (eczema) and acne vulgaris are being treated, including in the composition a medication or other ingredient that assists in such treatment can be advantageous; i.e., for example a medication or other ingredient that can provide a benefit upon rupture, removal, etc., of the biofilm by the solvent and solute components.

Non-limiting categories of medications that can be added to the composition include
steroids such as hydrocortisone, clobetasol propionate, betamethasone dipropionate, halobetasol propionate, diflorasone diacetate, fluocinonide, halcinonide, amcinonide, desoximetasone, triamcinolone acetonide, mometasone furoate, fluticasone propionate, betamethasone dipropionate, halometasone, fluocinolone acetonide, hydrocortisone valerate, hydrocortisone butyrate, flurandrenolide, triamcinolone acetonide, mometasone furoate, fluticasone propionate, desonide, fluocinolone acetonide, hydrocortisone valerate, alclometasone dipropionate, triamcinolone acetonide, fluocinolone acetonide, and desonide;
antibiotics such as Amikacin, Amoxicillin, Ampicillin, Arsphenamine, Azithromycin, Azlocillin, Aztreonam, Bacitracin, Capreomycin, Cefaclor, Cefadroxil, Cefalexin, Cefamandole, Cefazolin, Cefdinir, Cefditoren, Cefepime, Cefixime, Cefmetazole, Cefonicid, Cefoperazone, Cefotaxime, Cefotetan, Cefoxitin, Cefpodoxime, Cefprozil, Ceflaroline, Ceftazidime, Ceftibuten, Ceftizoxime, Ceflobiprole, Ceftriaxone, Cefuroxime, Cephalosporins, Cephalothin, Cephapirin, Cephradine, Chloramphenicol, Ciprofloxacin, Clarithromycin, Clindamycin, Clofazimine, Colistin, Cycloserine, Dalbavancin, Dapsone, Daptomycin, Dicloxacillin, Doripenem, Doxycycline, Enoxacin, Ertapenem, Erythromycin, Ethambutol, Ethionamide, Fidaxomicin, Flucloxacillin, Fosfomycin, Furazolidone, Fusidic acid, gatifloxacin, Geldanamycin, gemifloxacin, Gentamicin, grepafloxacin, Halicin, Herbimycin, Inipenem/Cilastatin, Isoniazid, Kanamycin, levofloxacin, Lincomycin, Linezolid, lomefloxacin, Loracarbef, Mafenide, Malacidins, Meropenem, Metacycline, methicillin, Metronidazole, Mezlocillin, Minocycline, Moxalactam, moxifloxacin, Mupirocin, nadifloxacin, Nafcillin, Nalidixic acid, Neomycin, Netilmicin, Nitrofurantoin, norfloxacin, ofloxacin, Omadacycline, Oritavancin, Oxacillin, Oxazolidinones, Oxytetracycline, Paromomycin, Penicillin G, Penicillin V, Piperacillin, Piperacillin/tazobactam, Platensimycin, Polymyxin B, polypeptides, Posizolid, pyrazinamide, Quinupristin/Dalfopristin, Radezolid, Rifabutin, Rifampicin, Rifapentine, Rifaximin, Roxithromycin, Serotonin Syndrome, silver sulfadiazine, sparfloxacin, Spectinomycin, Spiramycin, Streptomycin, Sulfacetamide, Sulfadiazine, Sulfadimethoxine, Sulfamethizole, Sulfamethoxazole, Sulfanilimide, Sulfasalazine, Sulfisoxazole, Sulfonamido-chrysoidine, Tedizolid, Teicoplanin, Teixobactin, Telavancin, Telithromycin, temafloxacin, Temocillin, tetracycline, Thiamphenicol, Thrombocytopenia, Ticarcillin, Ticarcillin/clavulanate, Tigecycline, Tinidazole, Tobramycin, Torezolid, Trimethoprim, Trimethoprim/sulfamethoxazole, trovafloxacin, and Vancomycin;
anticoagulants such as heparin, Apixaban, Dabigatran, Edoxaban, Enoxaparin, Rivaroxaban, and warfarin;
clotting promoters such as aprotinin, epsilon-aminocaproic acid, aminomethylbenzoic acid, and tranexamic acid;
antifungals such as Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, miconazole, Omoconazole, Oxiconazole, Sertaconazole, Sulconazole, Tioconazole, Albaconazole, Efinaconazole, Epoxiconazole, fluconazole, Isavuconazole, Itraconazole, Posaconazole, Propiconazole, Ravuconazole, Terconazole, Voriconazole, Abafungin, amorolfin, butenafine, naftifine, terbinafine, Anidulafungin, Caspofungin, Micafungin, Aurones, benzoic acid, Ciclopirox, Flucytosine or 5-fluoro, cytosine, Griseofulvin, Haloprogin, Tolnaftate, undecylenic acid, Triacetin, Crystal violet, Orotomide, Miltefosine, potassium iodide, Nikkomycin, copper(II) sulfate, selenium disulfide, sodium thiosulfate, Piroctone olamine, Iodoquinol (diiodohydroxyquin), Acrisorcin, zinc pyrithione, and sulfur;
anesthetics such as lidocaine, benzocaine, butamben, dibucaine, oxybuprocaine, pramoxine, proxymetacaine and tetracaine; and
analgesics such as 2-(4-(2-methylpropyl)phenyl)propanoic acid (i.e., ibuprofen), capsaicin, diclofenac, lidocaine, methyl salicylate, and trolamine.

Such medications preferably are delivered in purified water. Because some of the aforementioned classes of medications, or certain species within a given class, can have limited solubility in water, delivery in an organic liquid (or a solution which includes an organic liquid) might be necessary or desirable. In such cases, the considerations regarding type and amount of such organic liquid(s) set forth above should be taken into account.

When adding one or more of such medications to the composition container, the solubility limits of the medications at the composition's temperature necessarily must be taken into account.

Prior to evacuation of the container contents, the container and those contents can be warmed. While such warming can assist in assuring that all solute components are fully dissolved, it also provides the side benefit of bringing the temperature of the composition closer to that of a patient's internal temperature. In view of the latter, the composition temperature of preferably is within 5° C. of the body temperature of the particular type of mammalian patient. (In extremely hot climates, bringing the temperature of the composition to within the desired range might require cooling rather than warming.)

Where a medication is to be introduced into the container prior to the container contents being evacuated, the aforementioned temperature adjustment can occur before or after introduction of the medication to the composition.

Where a composition is applied to and/or around previously implanted medical hardware in situ, for example to address an implanted medical device having thereon a biofilm (as indicated for example by a patient showing signs of PJI), the application occurs after approximation of the original surgical wound cavity made during/for the initial implantation of the medical device. There is no time beyond which the present compositions no longer can be used after approximation of the surgical wound cavity made during/for the initial implantation of the medical device.

After reopening the patient to expose the implanted device so as to allow the compositions to contact the implanted hardware in situ, the compositions can be introduced in any manner, including in all of the ways described in WO 2022/081737. Transferring the composition from the interior of the container to the surgical wound cavity of the patient can be accomplished in numerous ways.

One option involves decanting the contents of the container into a sterile basin by means of a tube with a spiked end. Evacuation of container contents typically occurs solely through the force of gravity. Once decanted, a medical professional, e.g., surgeon, can pour the decanted composition from the basin over and into the wound cavity.

A variation of the foregoing involves use of a bulb syringe (or similar) by the medical professional to better direct flow of the composition into and around the wound cavity.

Where the container is a bottle (typically packaged in a thermoformed polymeric tray with a removable, polymeric lid), its contents can be evacuated similarly to the option just described. If the bottle is sealed, the seal is removed and a cap with nozzle applied. (If the bottle includes an integrated nozzle, this step can be avoided.) The medical professional can use the nozzle to direct composition flow into and around the wound cavity similarly to the manner employed with a bulb syringe.

Another option involves use of a device that can deliver the composition under pressure, e.g., a pulsed or jet lavage delivery system such as Interpulse™ pulsed lavage system (Stryker, Kalamazoo, Michigan) or Pulsavac™ Plus lavage system (Zimmer Biomet; Warsaw, Indiana). Similar to the gravity feed option described above, the composition can be accessed using a tube with a spiked end, with the other end of that tube being attached to and feeding the delivery instrument. A medical professional using the wand or gun portion of the delivery instrument directs flow of the composition into and rinses the surgical wound cavity.

Regardless of how introduced, the amount of composition delivered into the surgical wound cavity can vary from as little as a few milliliters for small surgical sites up to 0.5, 1, 1.5 or 2 L (optionally delivered in more than one aliquot).

Dwell times can vary within wide limits, from seconds to hours. Exemplary dwell times range from 30, 60, 120, 180, 240, 300, 360, 420, 480, 540, 600, 900, 1200, 1500, 1800, 3600, 4800, etc., seconds. Dwell times can be adjusted as desired, for example by measuring colony forming units (CFUs) during application contact of the composition, particularly where the surface being treated is inanimate.

The amount of composition used in the described methods is not limited. For example, a given surface can simply be wholly or partially covered/coated/contacted with the composition, which can then be left to act for a given dwell time. In other embodiment the composition can be continuously or intermittently reapplied to the surface of interest, thereby providing fresh composition to the surface being treated. The surface being treated, after application of the composition, can optionally be left to stand in air, can optionally be covered with a covering that decreases the evaporation of the composition, etc. If desired, the surface being treated can be rubbed, abraded, scrubbed, etc., during the dwell time to assist, increase, and/or speed up bioburden reduction.

In all instances herein, whether a composition is described as being applied to a surface or applied to a biofilm on a surface, or whether the application is described as a biofilm on a surface being contacted with a composition, typically both the surface and the biofilm are brought into intimate physical contact with the composition due to noncomplete surface coating by the biofilm.

Regardless of when and how used, in a preferred embodiment the composition does not require rinsing or suctioning. Some or all can remain at the site of the medical hardware, whether or not treatment involves a surgical procedure.

In the process of treating a biofilm on and/or around a previously implanted medical device in situ, at least a portion of the introduced composition typically remains behind after approximation of the surgical wound, similar to that which is described in the aforementioned WO 2022/081737, albeit here after treatment of an existing biofilm. The amount of remaining composition can be as little as necessary to provide a coating on exposed (internal) tissues (0.5 to 10 mL) to a significant percentage of the volume of composition introduced during treatment. The fact that some composition remains behind means that it can work to reduce bioburden after biofilm disruption and during the process of approximation and until such portion is biosorbed.

In situations where a not insubstantial amount of composition is introduced to the surgical wound cavity during the treatment procedure (e.g., ˜100 mL or more), partial removal via suction can be preferred. In situations where a substantial amount of composition is introduced during the procedure (e.g., ˜250 mL or more), partial removal via suction is preferred. (Some composition might exit via normal outflow during or after introduction.)

Where the treatment and composition introduction involves surgical incision and retraction, use of pressure delivery (e.g., pulsed lavage) systems is common, and many of those devices have integrated suctioning, i.e., the same device that introduces the composition is designed to also remove it with suction.

The amount of composition that remains in the surgical wound cavity during and after wound approximation typically ranges from a few milliliters up to ˜250 mL, with the amount largely depending on whether partial removal via suction has been employed.

Importantly, removal by suction need not be preceded and/or followed by a saline solution rinse, i.e., the composition is sufficiently gentle and biocompatible that its continued presence does not result in significant deleterious effects.

When wounds are closed, edges of the wound are approximated by standard techniques including sutures, staples, adhesive(s) and the like. Approximation can be complete or partial, e.g., incorporation of a wound drain.

After treatment, the area of and surrounding composition introduction can be rinsed with a disinfecting solution and/or covered with a sterile protecting layer (optionally with an antimicrobial gel or cream such as SURGX™ sterilized gel (Next Science; Jacksonville, Florida).

In preferred embodiments, the compositions are not diluted or removed, in part or in whole, from the area around and nearby the medical device and allow treatment of the biofilm thereon subsequent to cessation of the treatment.

The types of implanted medical devices that can be treated with the compositions in accordance with the methods disclosed herein are not limited, and include a screw, a pin, a wire, a rod, a plate, or an artificial joint component.

As discussed above, compositions described herein advantageously reduce bioburden in existing biofilms, wherever located. Alternatively or additionally, loss of integrity in the protective EPS/ECPS due to exposure to the composition can result in some or all of the biofilm being dissolved, washed away, or otherwise removed.

Assaying a change in bacterial CFUs before and after treatment with the composition can quantify a change in the number of viable bacteria (when exposure to the composition results in bacteria being killed) and/or reflect the loss of such bacteria, regardless of whether or not killed) due to reduction in size of the biofilm. When quantified by a change in CFU, the reduction in bioburden preferably is at least 90% (1 log), more preferably at least 99% (2 log), even more preferably at least 99.9% (3 log), and still more preferably at least 99.99% (4 log).

The following embodiments are specifically contemplated. An embodiment relating to a method of use involving a composition is intended to be read as also relating to the composition for use in that method.

Embodiment [1] relates to a method for treating a wound cavity in a mammalian subject, the method comprising:
a) providing a sterile liquid composition having an effective solute concentration of from 0.3 to 0.7 Osm/L and a pH of from 3.7 to 4.2, the composition consisting of solvent and solute components;
b) prior to approximation of the wound, introducing the composition to the wound cavity; and
c) permitting at least a portion of the composition to reduce bioburden in the wound cavity during and after approximation of the wound.

Embodiment [2] relates to the method of Embodiment [1] wherein the solvent component consists of purified water.

Embodiment [3] relates to the method of any preceding Embodiment wherein the solute component comprises from 0.7 to 1.9 g/L ionic surfactant.

Embodiment [4] relates to the method of any preceding Embodiment wherein the providing step involves delivery of the composition in a container that comprises at least one access point.

Embodiment [5] relates to the method of any preceding Embodiment wherein the solute component consists of a buffer system and an ionic surfactant.

Embodiment [6] relates to a process for treating a wound cavity in a mammalian subject, the process comprising:
a) providing a container that comprises at least one access point that holds a sterile liquid composition having an effective solute concentration of from 0.3 to 0.7 Osm/L and a pH of from 3.7 to 4.2, the composition consisting of
   1) a solvent component consisting of purified water, and
   2) a solute component comprising a buffer system and from 0.7 to 1.9 g/L ionic surfactant,
b) prior to approximation of the wound, introducing the composition to the wound cavity; and
c) permitting at least a portion of the composition to reduce bioburden in the wound cavity during and after approximation of the wound.

Embodiment [7] relates to the method of any of Embodiments [4] to [6] wherein the container comprises multiple access points, the method further comprising adding at least one medication to the composition prior to the introducing step.

Embodiment [8] relates to the method of any of Embodiments [5] to [7] wherein the buffer system comprises dissociation products of a carboxylic acid and a conjugate base of a carboxylic acid.

Embodiment [9] relates to the method of Embodiment [8] wherein the buffer system consists of dissociation products of at least one carboxylic acid and at least one conjugate base of at least one carboxylic acid.

Embodiment [10] relates to the method of any of Embodiments [8] to [9] wherein the carboxylic acid is citric acid and wherein the conjugate base is a citrate.

Embodiment [11] relates to the method of any preceding Embodiment wherein the composition has an effective solute concentration of from 450 to 680 mOsm/L.

Embodiment [12] relates to the method of any preceding Embodiment wherein the composition pH is from 3.85 to 4.05.

Embodiment [13] relates to the method of any of Embodiments [5] to [12] wherein the ionic surfactant is an anionic surfactant.

Embodiment [14] relates to the method of any preceding Embodiment wherein the composition has an effective solute concentration of from 350 to 590 mOsm/L.

Embodiment [15] relates to a method for treating a surgical site in a mammalian subject, the method consisting of:
a) providing a container that comprises at least one access point that holds a sterile liquid composition having an effective solute concentration of from 450 to 675 mOsm/L and a pH of from 3.85 to 4.05, the composition consisting of
   1) a solvent component consisting of purified water, and
   2) a solute component that consists of
      (A) a buffer system that comprises dissociation products of citric acid and at least one citrate,
      (B) from 0.75 to 1.25 g/L anionic surfactant, and
      (C) optionally, one or more adjuvants selected from dyes, preservatives and viscosifiers;
b) where the container comprises more than one access point, optionally adding at least one medication to the composition;
c) prior to approximation of the surgical site opening, introducing the composition to the opening; and
d) permitting at least a portion of the composition to reduce bioburden at the surgical site during and after approximation.

Embodiment [16] relates to the method of Embodiments [15] wherein the at least one citrate consists of trisodium citrate.

Embodiment [17] relates to the method of and preceding Embodiment wherein the composition pH is 3.95±0.05.

Embodiment [18] relates to the method of any preceding Embodiment wherein the composition is undiluted prior to wound approximation.

Embodiment [19] relates to the method of any preceding Embodiment wherein a portion of the composition is removed or diluted prior to the wound approximation.

Embodiment [20] relates to the method of any of Embodiments [6] and [8]-[19] wherein the buffer system comprises dissociation products of from 25 to 40 g/L citric acid and from 30 to 45 g/L of a citrate that comprises three alkali metal ions.

Embodiment [21] relates to the method of Embodiments [20] wherein the buffer system comprises dissociation products of from 30 to 38 g/L citric acid and of from 34 to 42 g/L of trisodium citrate.

Embodiment [22] relates to the method of any preceding Embodiment wherein all solutes in the solute component are pharmaceutical grade.

Embodiment [23] relates to the method of any preceding Embodiment further comprising, prior to the introducing step, providing the container at a temperature that is within 5° C. of the body temperature of the mammalian subject or adjusting the temperature of the composition to within 5° C. of the body temperature of the mammalian subject.

Embodiment [24] relates to the method of any preceding Embodiment wherein the composition is introduced by an emergency medical service provider, wherein at least some of the bioburden reduction occurs prior to or during transport of the mammalian subject.

Embodiment [25] relates to the method of any preceding Embodiment wherein the composition is introduced during an operation in a surgical theater, wherein the bioburden reduction occurs before, during and after the wound approximation.

Embodiment [26] relates to the method of any of Embodiments [13] to [25] wherein the anionic surfactant is sodium lauryl sulfate.

Embodiment [27] relates to the method of any preceding Embodiment wherein the composition has an effective solute concentration of 525±50 mOsm/L.

Embodiment [28] relates to the method of Embodiment [27] wherein the effective solute concentration is 525±25 mOsm/L.

Each aspect, embodiment, feature, etc., of the invention described herein, whether or not preferred, can be used with, combined with, etc., any one or more other described aspect, embodiment, feature, etc., of the invention described herein, whether or not preferred.

When an amount, concentration, or other value or parameter is given as a range, or as a list of values, this is to be understood as including endpoints, as specifically disclosing all ranges formed from any pair of any upper and lower values, and as specifically disclosing all integers and fractions within the range, regardless of whether ranges, all integers, and fractions are separately disclosed. For example, an amount, concentration, or other value or parameter given as a range of 3-10, or as a listing of the values 3, 6, 7, 9, and 10, both specifically disclose and include the range 5-7 and 6-9, and the value 4.7.

1: A method for in situ treatment of an implanted medical device having thereon a biofilm, said method comprising contacting said biofilm with a liquid composition comprising solvent and solute components and having a calculated effective solute concentration of from 0.3 to 1.4 Osm/L and a pH of from 3.7 to 4.2 for a time sufficient to reduce surface or skin bioburden resulting from said biofilm.

2: The method of claim 1 wherein said composition has a calculated effective solute concentration of from 350 to 590 mOsm/L.

3: The method of claim 2 wherein said composition has a calculated effective solute concentration of 525±50 mOsm/L.

4: The method of claim 1 wherein said composition has a calculated effective solute concentration of from 450 to 680 mOsm/L.

5: The method of claim 1 wherein said composition has a pH of from 3.85 to 4.05.

6: The method of claim 5 wherein said pH is 3.95±0.05.

7: The method claim 1 wherein said solvent component consists of purified water.

8: The method of claim 1 wherein said solute component comprises a buffer system and an ionic surfactant.

9: The method of claim 8 wherein said buffer system consists of dissociation products of at least one carboxylic acid and at least one conjugate base of at least one carboxylic acid.

10: The method of claim 9 wherein said buffer system comprises dissociation products of from 25 to 40 g/L citric acid and from 30 to 45 g/L of a citrate that comprises three alkali metal ions.

11: The method of claim 8 wherein said composition comprises from 0.7 to 1.9 g/L ionic surfactant.

12: The method of claim 11 wherein said ionic surfactant is an anionic surfactant.

13: The method of claim 1 wherein said liquid composition is sterile.

14. (canceled)

15: The method of claim 1 wherein said solute component comprises a buffer system that comprises dissociation products of citric acid and at least one citrate.

16: The method of claim 15 wherein said buffer system comprises dissociation products of from 30 to 38 g/L citric acid and of from 34 to 42 g/L of trisodium citrate.

17: The method of claim 16 wherein the liquid composition has a pH gf 3.95±0.1.

18: The method of claim 15 wherein said solute component further comprises from 0.75 to 1.25 g/L anionic surfactant.

19. (canceled)

20: The method of claim 1 wherein an implanted medical device having a biofilm thereon is the object of in situ treatment, said solute component of said composition comprising anionic surfactant.

21: The method of claim 22 wherein said composition is applied to the skin of a patient suffering from atopic dermatitis or acne vulgaris.

22: A method for treating human skin having thereon a biofilm, said method comprising contacting said biofilm with a liquid composition comprising solvent and solute components and having a calculated effective solute concentration of from 0.3 to 1.4 Osm/L and a pH of from 3.7 to 4.2 for a time sufficient to reduce surface or skin bioburden resulting from said biofilm.
